(19) United States
(12) Patent Application Publication
(10) Pub. No.: US 2007/0196244 A1
(43) Pub. Date: Aug. 23, 2007

(54) Air/water sterilization system for ice machine

(76) Inventor: Carlton R. Croft, Eaton Park, FL (US)

Correspondence Address: LITMAN LAW OFFICES, LTD., P.O. BOX 15035, CRYSTAL CITY STATION, ARLINGTON, VA, 22215, US

(21) Appl. No.: 11/708,062

(22) Filed: Feb. 20, 2007

Related U.S. Application Data
(60) Provisional application No. 60/775,338, filed on Feb. 22, 2006.

(51) Int. Cl. A61L 9/20

(52) U.S. Cl. 422/121; 250/435; 96/224

(57) ABSTRACT

The air-and-water sterilization apparatus for use with an ice machine includes a source of ultraviolet radiation, a first input, a first output, a water tube, an air chamber, an air treatment passageway, a second input, a first filter, a first damper, a third input, a second damper, a second filter, a fan, a second output, and a housing. The source of ultraviolet radiation is one or more ultraviolet lamps that are used to sterilize air and water before that are moved off to the ice machine or other appliance. The source of ultraviolet radiation, the water tube, the. air chamber, the air treatment passageway, the first filter, the first damper, the second damper, the second filter, and the fan are all encompassed in the housing to create a protected and compact sterilization apparatus

### CROSS-REFERENCE TO RELATED APPLICATION

This application claims the benefit of U.S. Provisional Patent Application Ser. No. 60/775,338, filed Feb. 22, 2006.

### BACKGROUND OF THE INVENTION

1. Field of the Invention

The present invention relates to sterilization systems, and more specifically to an air and water sterilization apparatus for use with appliances such as ice machines and the like.

2. Description of the Related Art

Ice machines generally consist of an icemaker and a compartment where the ice is stored for retrieval. Ice machines are usually connected to a water source, typically a utility line, so that ice can replenish as quantities are dispensed from the compartment. One problem with this current arrangement is that ice made from tap water is not free from contaminants. Another problem arises in that the ice stored in the compartment is surrounded by stale air, which stale air has been contaminated by outside factors.

Therefore, there is a need for an apparatus that can be coupled with an ice machine to provide sterilized water and sterilized air to the icemaker and ice compartment components of the ice machine. It is desirable that the apparatus be able to expose both incoming water and air to a sterilizing force for an extended period of time before releasing them into the ice machine. It is also desirable that the apparatus be capable of being used for the sterilization of water and/or air in other common appliances. Thus an air and water sterilization system for an ice machine solving the aforementioned problems is desired.

There are many devices in the related art that are adapted to generate ice for dispensing. Pertinent examples of such art have been cited and identified in the accompanying IDS. However, none of the cited and identified related art, taken either singly or in combination, is seen to disclose an air and water sterilization system for an ice machine or the like as will subsequently be described and claimed in the instant invention.

### SUMMARY OF THE INVENTION

The present invention is an air and water sterilization apparatus for use with an ice machine. The apparatus incorporates a source of ultraviolet radiation, a first input, a first output, a water tube, an air chamber, an air treatment passageway, a second input, a first filter, a first damper, a third input, a second damper, a second filter, a fan, a second output, and a housing. The source of ultraviolet radiation includes one or more ultraviolet lamps. The ultraviolet radiation functions to sterilize air and water before they are injected into the ice machine or other appliance. A control system is employed to correlate the number of lamps used as a function of the amount of ice present in the ice chamber.

Accordingly, the invention presents a fluid sterilization apparatus that greatly improves the quality of ice retrieved from an ice-making machine and the like. The invention provides for improved elements and arrangements thereof for the purposes described which are inexpensive, dependable and fully effective in accomplishing their intended purposes.

A clear understanding of the present invention will become readily apparent upon further review of the following specification and drawings.

### BRIEF DESCRIPTION OF THE DRAWINGS

FIG. 1 is an environmental, perspective view of an air-and-water sterilization system for use with an ice machine according to the present invention.

FIG. 2 is an exploded, perspective view of an air-and-water sterilization system for use with an ice machine according to the present invention.

FIG. 3 is a perspective view of an air-and water-sterilization apparatus according to the present invention.

FIG. 4 is a side, sectional view of an air-and-water sterilization apparatus according to the present invention with a portion of the housing removed, exposing the internal features.

FIG. 5 is a side, sectional view of a second embodiment an air-and-water sterilization apparatus according to the present invention with a portion of the housing removed, exposing the internal features.

FIG. 6 is a plan view of an air sterilization apparatus according to the present invention with a portion of the housing removed, exposing the internal features.

FIG. 7 is a schematic view for a control system that is used to control the ultraviolet lamps

Similar reference characters denote corresponding features consistently throughout the attached drawings.

### DETAILED DESCRIPTION OF THE PREFERRED EMBODIMENTS

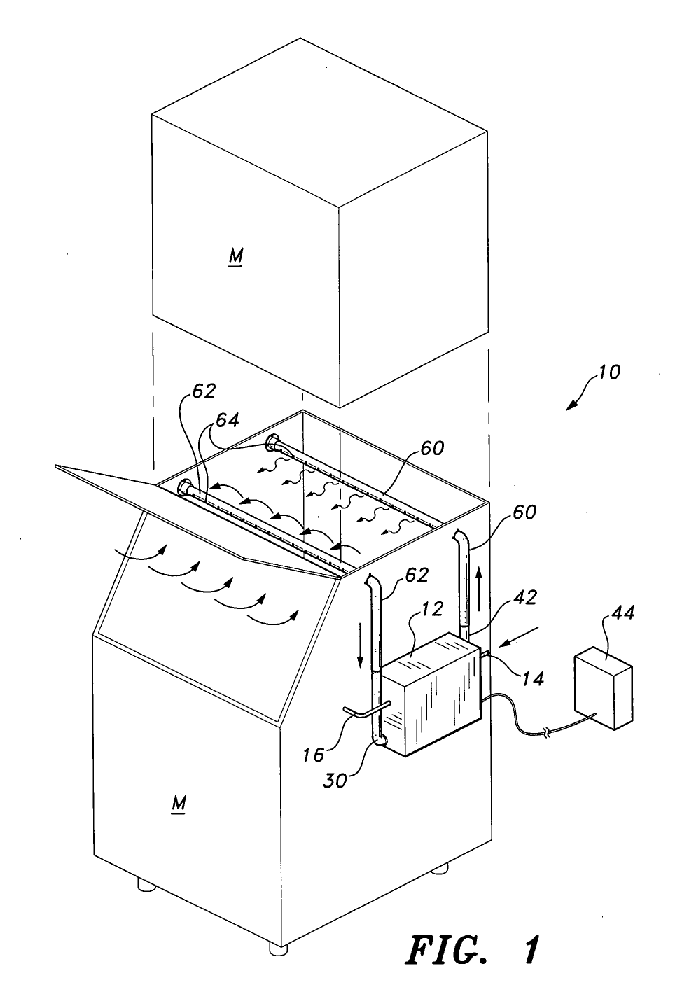

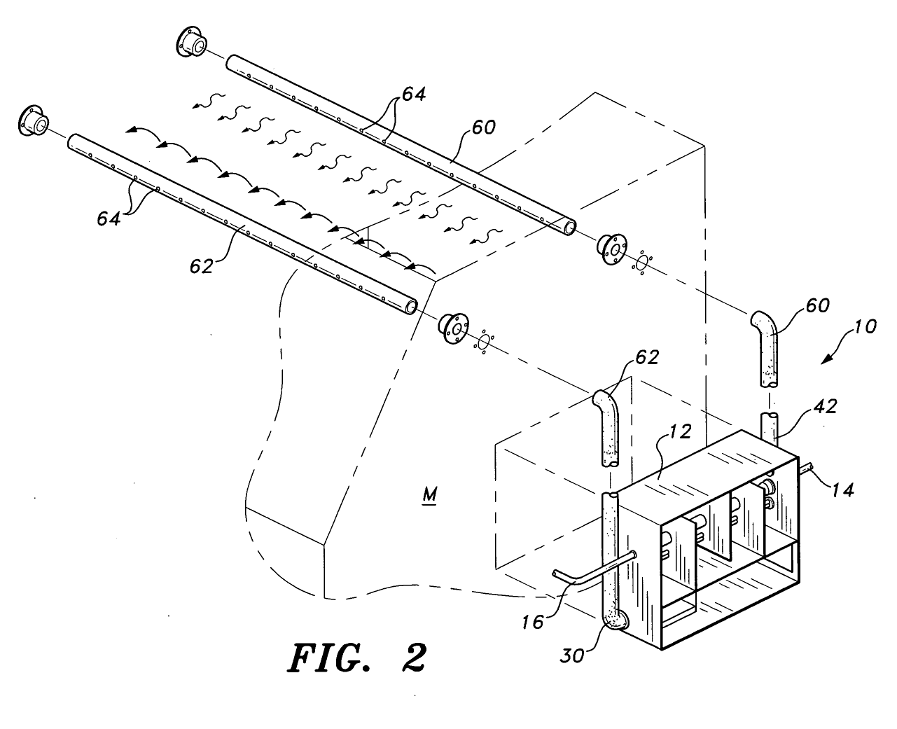

Attention is first directed to FIGS. 1 and 2 of the drawings wherein the air-and-water sterilization apparatus is generally indicated at 10 and is shown in use with an ice machine M. Air-and-water sterilization apparatus 10 is mostly contained within housing 12 and is powered by an external power source 44. External water for use in ice machine M is introduced into the air-and-water sterilization apparatus 10 from the water source, typically a utility line, through first input 14. Once the water has been sterilized, it is exited from air-and-water sterilization apparatus 10 through first output 16. Return air from ice machine M is received into air-and-water sterilization apparatus 10 through third input 30 and the treated and sterilized air is exited from air-and-water sterilization apparatus 10 through second output 42.

In a preferred embodiment, stale air from inside the ice holding compartment of ice machine M and outside air captured from outside through the door of ice machine M are gathered by intake tube 62 through apertures 64. Intake tube 62 carries this combined contaminated air to third input 30 where it can be introduced into air-and-water sterilization apparatus 10 for treatment. Once the air is treated, it is released from air-and-water sterilization apparatus 10 through second output 42 and carried back into the ice holding compartment of ice machine M through outtake tube 60 and released through apertures 64.

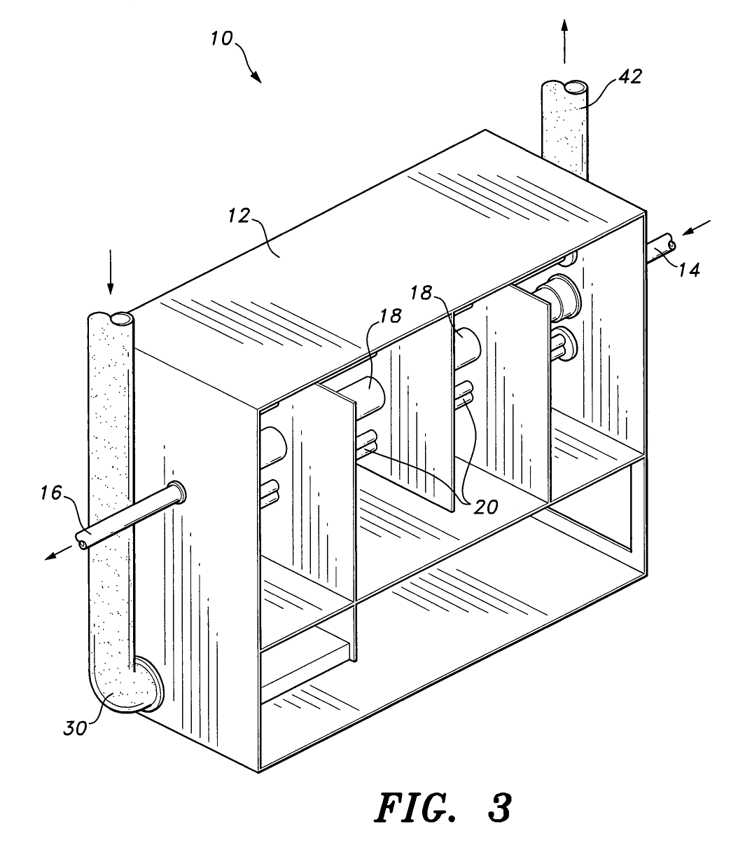

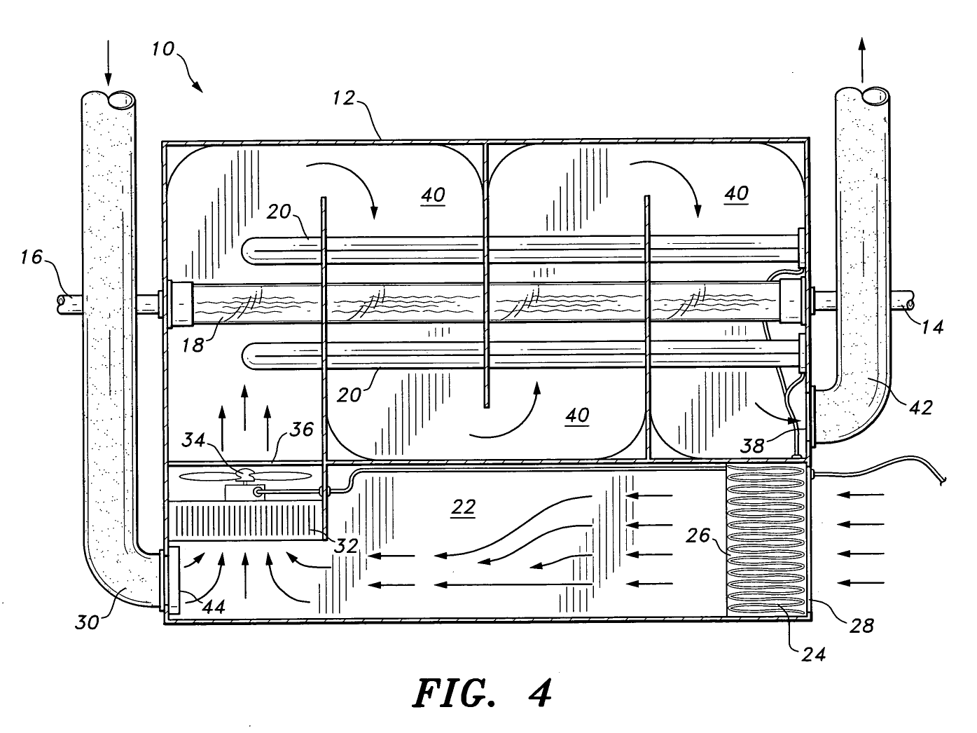

Referring to FIGS. 3 and 4 of the drawings, air-and-water sterilization apparatus 10 is shown with ultraviolet lamps 20, water tube 18, air chamber 22, air treatment passageway 40, first filter 24, first damper 26, second damper 44, second filter 32, and a fan 34 within housing 12. Water tube 18 is connected between first input 14 and first output 16 and carries water through the system such that it is exposed to the radiation from ultraviolet lamps 20 and sterilized. In a preferred embodiment, water tube 18 is made from quartz glass, thereby maximizing the water's exposure to the radiation as it is moved through the system.

Air chamber 22 is used to collect air that is to be treated from two different sources, second input 28 and third input 30. Second input 28 receives ambient air from outside of the system and processes it through first filter 24 before it arrives in air chamber 22. First damper 26 is used to control the flow of ambient air into the system such that the system does not receive too much or too little air to process at any given time. Third input 30 receives return air from the ice machine or other appliance that is being used in conjunction with air-and-water sterilization apparatus 10. The flow of the return air, into air chamber 22, is controlled by second damper 44.

Once a certain amount of air has been combined and gathered in air chamber 22, it is passed through second filter 32. In a preferred embodiment, second filter 32 is an electro-static plate filter. After the air has been filtered through second filter 32, it is pushed by fan 34 through the entrance 36 and on throughout air treatment passageway 40. Air treatment passageway 40 is a sinusoidal-shaped passageway that allows the air to be exposed to radiation from ultraviolet lamps 20 along its entire path through the system. The winding, sinusoidal configuration of the air treatment passageway 40 allows the air to be exposed to the radiation for an extended period of time, resulting in cleaner and healthier air than what is produced by other “straight line” type systems. The surfaces of the air treatment passageway 40 are coated with titanium dioxide to promote photo-catalytic oxidation. The reaction of ultraviolet light and the coated surfaces functions to maintain ozone production at safe levels. Once the air has traveled the entire length of air treatment passageway 40 and arrived at exit 38, it is evacuated from the system by second output 42, which, in turn, can be used to carry the air back to the ice machine or other appliance that is being used in conjunction with the air-and-water sterilization apparatus 10.

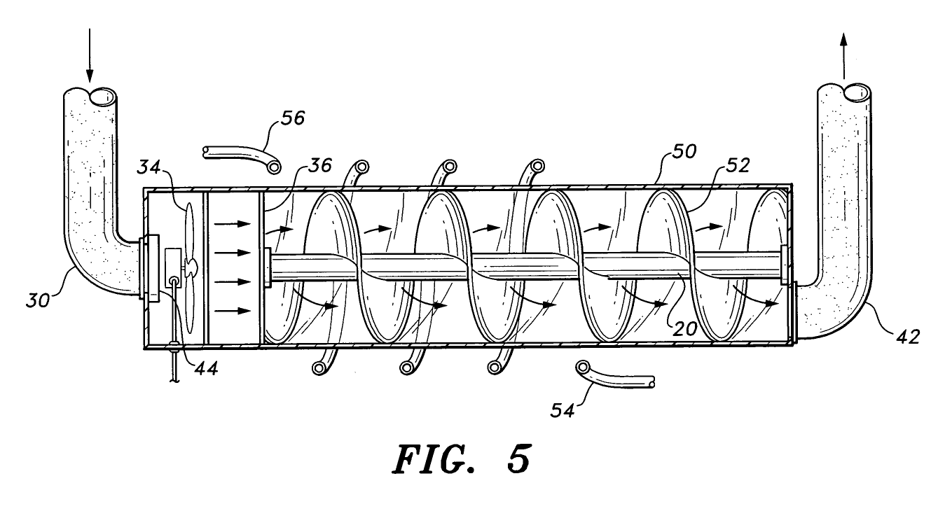

It is understood that air treatment passageway 40 may be configured in other ways that will allow the air to be exposed to the radiation for an extended period of time, as opposed to other “straight line” type systems. For example, FIG. 5 is illustrative of an embodiment that employs an air chamber 50 having a wall 52 therein that is configured to direct the air in a spiral path. One or more ultraviolet lamps 20 extend along the center of wall 52. A water tube having water inlet 54 and water outlet 56 is coiled around the outer surface of air chamber 50. The wall of chamber 50 and will be transparent to ultraviolet rays in the vicinity of the water tube. The wall 52 is also coated with titanium oxide.

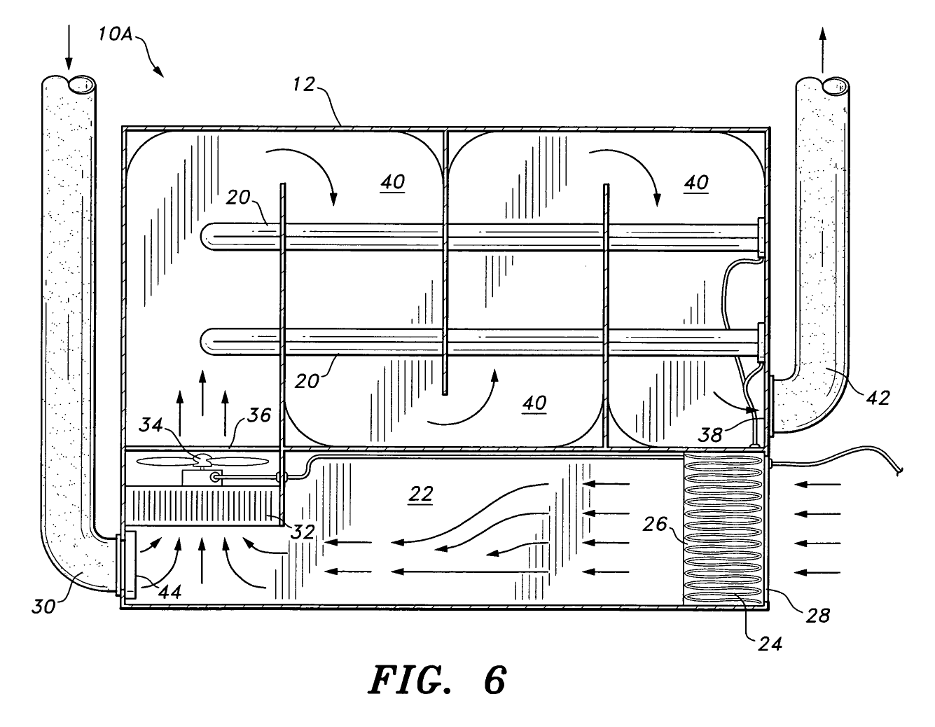

FIG. 6 shows an alternate embodiment of the apparatus that eliminates the water treatment aspect and employs only an air sterilization apparatus 10A. Air sterilization apparatus 10A treats only air in a fashion identical to that described above.

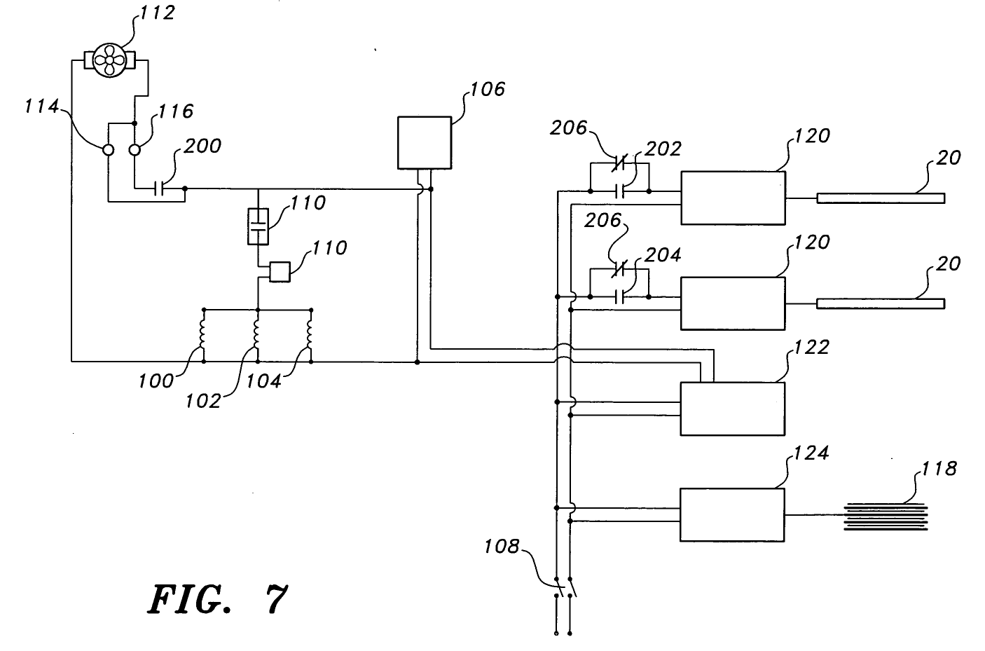

FIG. 7 displays a schematic for a control system that is used to control the source of ultraviolet radiation, namely, ultraviolet lamps 20. The control system is connected between the air-and-water sterilization apparatus and the ice machine and includes ice level detection device 110, relays 100, 102, and 104, timer 106, variable resistors 114 and 116, ice machine fan 112, switch 108, electronic air filter 118, electronic air filter power supply 124, DC power supply 122, and ballasts 120. Switch 118 is an on/off switch that controls the flow of AC to the control system. The AC powers electronic air filter power supply 124, DC power supply 122, and ballasts 120. Timer 106 is used for powering on/off DC power supply 122, which in turn provides power to fan 112 and relays 100, 102, and 104. Electronic air filter 118 is provided for filtering air within the system and helps to pressurize the air inside the ice compartment of the ice machine to a level greater than the pressure of the air outside of the compartment. Ice level detection device 110, such as a thermostat and bulb combination or a sonar device, determines the level of ice within the ice compartment. When the ice level is low, ice level detection device 110 signals the control system to control fan speed and determine whether one of the UV lamps 20 needs to be dropped out. This results in relay 100, which is in contact with fan 112 at contact 200, signaling ice machine fan 112 to increase production. Also, relays 102 and 104, which are in contact with ballasts 120 at 202 and 204, signal ballasts 120 to operate both UV lamps 20 as a result of the increased amount of air and water that will need to be sterilized during increased production. At other times, when the ice level is higher, the control system conserves power by operating only one UV lamp 20 at a time, allowing the lamps to alternate use in order to preserve their lifetimes equally.

It is to be understood that the present invention is not limited to the embodiments described above, but encompasses any and all embodiments within the scope of the following claims.

I claim:

1. An air-and-water sterilization apparatus for use with an appliance, comprising:
a housing
a source for emitting ultraviolet radiation disposed in said housing;
a tube containing water mounted on said housing said tube being exposed to said source for emitting ultraviolet radiation;
structure for directing water into and out of said tube;
an air passageway disposed in said housing, said air passageway being exposed to said source for emitting ultraviolet radiation; and
apparatus for directing air through said air passageway.

2. The air-and-water sterilization apparatus according to claim 1, wherein said apparatus for directing air through said air passageway includes an air chamber disposed adjacent said air passageway.

3. The air-and-water sterilization apparatus according to claim 1, wherein said source for emitting ultraviolet light includes at least one ultraviolet lamp.

4. The air-and-water sterilization apparatus according to claim 1, wherein said air passageway defines a sinusoidal flow path.

5. The air-and-water sterilization apparatus according to claim 1, wherein said air passageway defines a spiral flow path.

6. The air-and-water sterilization apparatus according to claim 1, wherein said air passageway is coated with a compound that promotes photo-catalytic oxidation.

7. The air-and-water sterilization apparatus according to claim 1, wherein said appliance is an ice making machine.

8. An air-and-water sterilization apparatus for use with an appliance, comprising:
a housing
at least one lamp for emitting ultraviolet radiation disposed in said housing;
a tube containing water mounted on said housing said tube being exposed to said at least one lamp for emitting ultraviolet radiation;
structure for directing water into and out of said tube;
an air treatment passageway disposed in said housing, said air treatment passageway being exposed to said at least one lamp for emitting ultraviolet radiation;
apparatus for directing air through said air treatment passageway, said apparatus for directing air including an air chamber disposed adjacent said air treatment passageway; and
a fan disposed in said air chamber, said fan causing the air to flow from said air chamber through said air treatment passageway.

9. The air-and-water sterilization apparatus according to claim 8, wherein said air treatment passageway is coated with a compound that promotes photo-catalytic oxidation and wherein air filters are disposed in said air chamber for filtering the air flowing therethrough.

10. The air-and-water sterilization apparatus according to claim 8, wherein said appliance is an ice making machine, said ice making machine having a compartment and further comprising:
an air return conduit extending from said compartment to said air chamber for returning stale air from said compartment, said air return conduit having apertures therein; and
an air supply conduit extending from said compartment to said air treatment passageway for supplying treated air to said compartment, said air supply conduit having apertures therein.

11. The air-and-water sterilization apparatus according to claim 10, wherein there are two lamps for emitting ultraviolet radiation and further comprising:
a control system including an ice level detection device electronically connected to said two lamps for controlling operation of each lamp relative to the ice level.

12. The air-and-water sterilization apparatus according to claim 11, wherein said air passageway defines a sinusoidal flow path.

13. The air-and-water sterilization apparatus according to claim 11, wherein said air passageway defines a spiral flow path.

14. An air sterilization apparatus for use with an appliance, comprising:
a housing
a source for emitting ultraviolet radiation disposed in said housing;
an air treatment passageway disposed in said housing, said air treatment passageway being exposed to said source for emitting ultraviolet radiation, said air treatment passageway being coated with titanium dioxide; and
apparatus for directing air through said air passageway.

15. The air sterilization apparatus according to claim 14, wherein said apparatus for directing air includes an air chamber disposed adjacent said air treatment passageway; and
a fan disposed in said air chamber, said fan causing the air to flow from said air chamber through said air treatment passageway.

16. The air sterilization apparatus according to claim 15, including air filters disposed in said air chamber.

17. The air-and-water sterilization apparatus according to claim 15, wherein said air passageway defines a sinusoidal flow path.

18. The air-and-water sterilization apparatus according to claim 15, wherein said air passageway defines a spiral flow path.
